SEQUENCE LISTING (4 sequences)

SEQ ID NO: 1 - DNA, 16 bp, Artificial Sequence
  misc_feature (1)..(16): Granulysin primer
tctctcgtctgagccc

SEQ ID NO: 2 - DNA, 17 bp, Artificial Sequence
  misc_feature (1)..(17): Granulysin primer
gcagcattggaaacact

SEQ ID NO: 3 - DNA, 16 bp, Artificial Sequence
  misc_feature (1)..(16): Actin Primer
acatccgcaaagacct

SEQ ID NO: 4 - DNA, 17 bp, Artificial Sequence
  misc_feature (1)..(17): Actin Primer
agggtgtaacgcaacta

(19) United States
(12) Patent Application Publication
(10) Pub. No.: US 2018/0312922 A1
(43) Pub. Date: Nov. 1, 2018

(54) METHOD AND IDENTIFICATION KIT FOR IDENTIFYING CAUSATIVE DRUG FOR DRUG HYPERSENSITIVITY REACTION

(71) Applicant: Chang Gung Memorial Hospital, Linkou, Taoyuan City (TW)

(72) Inventors: Wen-Hung CHUNG, Guishan Dist. Taoyuan City (TW); Shuen-Iu HUNG, Guishan Dist. Taoyuan City (TW)

(21) Appl. No.: 15/771,891

(22) PCT Filed: Oct. 30, 2015
(86) PCT No.: PCT/CN2015/093319

§ 371 Date: Apr. 27, 2018

(51) Int. Cl. C12Q 1/6883; G01N 33/68

(52) U.S. Cl. CPC C12Q 1/6883; C12Q 2600/136; C12Q 2600/158; G01N 33/6893

(57) ABSTRACT

Disclosed are a method and an identification kit for identifying a causative drug for a drug hypersensitivity reaction, including: first, biological sample lymphocytes and a suspected drug or the metabolites thereof are co-cultured in vitro to form reactants, and then the expression levels of the granulysin protein, polypeptide or mRNA in the reactants are detected and compared with the control values, then the degree that the suspected drug or the metabolites thereof activate lymphocytes can be identified, thus identifying the causative drug that initiates the drug hypersensitivity reaction. The causative drug comprises Western medicine, traditional Chinese medicine, a vaccine and an antigen molecule that can cause T-cell activation.

### BACKGROUND OF THE INVENTION

### 1. Field of the Invention

This invention relates to method and identification kit for identifying causative drugs of drug hypersensitivity reaction, including incubating the lymphocytes from a biological sample with the suspected causative drug or its metabolites in vitro to form a reactant, and quantify the expression level of granulysin protein, polypeptides or mRNA in the reactant and comparing to that of the control, to identify causative drugs of drug hypersensitivity reactions. The present invention has excellent properties such as a single, easy-to-execute technique, being quick, economic, and having a high sensitivity and high specificity and so on.

### 2. Description of the Related Art

Drug hypersensitivity reactions are a kind of drug-induced, potentially fatal immune disease, range from the milder forms of maculopapular eruption (MPE), erythema multiforme majus (EMM), fixed drug eruption (FDE) to life-threatening severe cutaneous adverse reactions (SCAR), which include drug rash with eosinophilia and systemic symptoms (DRESS), Stevens-Johnson syndrome (SJS), and toxic epidermal necrolysis (TEN).

SCAR is thought to involve drug-specific T cells. The conventional lymphocyte transformation test (LTT) is the most widely used to examine causative drugs of T cells-mediated hypersensitivity. It is an in vitro T cell activation and proliferation culture method by obtaining the lymphocytes from patients, stimulating the lymphocytes with suspected drugs, and measuring the amount of ³H-thymidine incorporation in DNA to determine the T lymphocyte proliferation during the one-week period of in vitro culture. However, the sensitivity of LTT is very low for SCAR, and examination of causative drugs of SJS/TEN usually yield negative results. In addition, the operation of radioactivity assay requires experienced technicians and costly instruments, the potential health threats from the radiation, only be practiced by licensed personnel and under approved radiation-operating environments are the limitations of this assay.

Another way to diagnose drug hypersensitivity reaction is by detecting the synthesis and expression level of cytokines, such as interferon gamma (IFN-γ), interleukin (IL)-2,5,13. This way involves the activation of T cells and evaluation the amount of cytokine in the culture supernatant, or the amount of intracellular cytokine via flow cytometry, or intracellular cytokine gene expression via polymerase chain reaction (PCR) or reverse transcription-PCR (RT-PCR). Although these cytokines could be used to identify the causative drugs from patients with drug hypersensitivity reactions, these cytokines are usually not specific for drug hypersensitivity and the sensitivity is also very low, therefore not suitable for clinical use. Moreover, this method requires more blood sample, the use of costly flow cytometer to detect the immune molecules on the surface of the blood cells, with low sensitivity, increased expenditure for purchasing reagents, and increased time and labor consumed.

The inventors first discovered that granulysin (GNLY) is a key mediator in the disseminated keratinocyte death in SJS/TEN. Our granted patent TW 1333978 (U.S. Pat. No. 7,718,378 B2) discloses the uses of granulysin in methods of diagnosing or treating SJS and TEN. However, the patent did not disclose the methods for identification of causative drugs of drug hypersensitivity reactions by granulysin.

In view of the above, conventional LTT is an expensive method with limited sensitivity. The practice of LTT requires experienced technicians with licenses for operating radioactive materials, and also limited by the approved radiation-operating environments. On the other hand, intracellular granulysin detection by cell cytometry has a lower sensitivity, and requires combination of other methods for identifying causative drugs of SCAR. At present, there is not yet a method with a high sensitivity, high reliability but low cost to identify causative drugs of drug hypersensitivity reactions. Therefore, there is still room for improvement of the prior technology.

### SUMMARY OF THE INVENTION

The present invention relates to a method for identification of causative drugs of drug hypersensitivity reactions, comprising the steps of: step 1: incubating the lymphocytes from a biological specimen with a suspected drug or its metabolite(s) in vitro to form a reactant; step 2: quantifying the expression level of granulysin protein, polypeptides or mRNA in the reactant and comparing it to that of the control. If the expression level of granulysin protein, polypeptides or mRNA is ≥1.2-fold higher compared to that of the control, the suspected drug or its metabolite(s) caused more lymphocyte activation and is the causative drug and probably induced the drug hypersensitivity reaction.

The term “control” refers to the granulysin expression level of lymphocytes from a biological specimen which are incubated in a culture medium with or without the solvent for the causative drug.

Wherein, the expression level of granulysin in step 2 is quantified by a capture antibody and a detection antibody specific to the granulysin protein or polypeptide in the reactant.

Wherein, the expression level of granulysin in step 2 is quantified by hybridizing the granulysin mRNA in the reactant with specific oligonucleotide primers or probes.

Wherein, the method for detecting the expression level of granulysin in the reactant by a capture antibody and a detection antibody is enzyme-linked immunosorbent assay (ELISA) or enzyme-linked immunospot (ELISPOT).

Wherein, lymphocytes include cells from peripheral blood or biological fluids isolated from a subject, preferably from peripheral blood.

Wherein, the drug hypersensitivity reactions comprise Stevens-Johnson syndrome, toxic epidermal necrolysis, drug rash with eosinophilia and systemic symptoms, maculopapular eruption, erythema multiforme majus, and fixed drug eruption.

Wherein, the causative drug includes western drugs, Chinese medicine, vaccines or antigens which can induce T cell activation.

An identification kit for identification of causative drug of drug hypersensitivity reactions, comprises the following reagents: a test kit, wherein the test kit co-cultivates a reagent and lymphocytes from a biological specimen with a suspected causative drug or its metabolite(s) in vitro to form a reactant; a detection kit, wherein the detection kit interacts with the reactant produced by the test kit. The expression level of granulysin protein, polypeptides or mRNA in the reactant is quantified and compared to that of the control. If the expression level of granulysin protein, polypeptides or mRNA is ≥1.2-fold higher compared to that of the control, the suspected drug or its metabolitea(s) is the causative drug.

Wherein, the detection kit comprises a specific capture antibody and a detection antibody which are specific for granulysin to quantify the expression level of granulysin in the reactant.

Wherein, the detection kit comprises oligonucleotide primers or probes which are specific for granulysin mRNA or genomic DNA to quantify the mRNA expression level of granulysin in the reactant.

Wherein, the method for detecting the expression level of granulysin in the reactant by the specific capture antibody and the detection antibody is enzyme-linked immunosorbent assay (ELISA) or enzyme-linked immunospot (ELISPOT).

Wherein, the drug hypersensitivity reactions comprise Stevens-Johnson syndrome, toxic epidermal necrolysis, drug rash with eosinophilia and systemic symptoms, maculopapular eruption, erythema multiforme majus, and fixed drug eruption.

Wherein, causative drug includes western drugs, Chinese medicine, vaccines or antigens which can induce T cell activation.

The present invention directs to incubating lymphocytes with a suspected drug or its metabolite(s) in vitro to form a reactant and the use of oligonucleotide primers, probes, and capture and detection antibodies against the granulysin which is produced by the activated lymphocytes. Wherein, the in vitro culture conditions include the concentrations of suspected drugs or its metabolites, the compositions of culture medium, and the incubation time. The present invention has excellent properties such as a single, easy-to-execute technique, being quick, economic, and having a high sensitivity and high specificity and so on.

With respect to the technology, the means and the effects of the present invention, it is believed that the above objects and features of the present invention can be better understood from the following detailed description when taken in conjunction with the accompanying drawings.

### BRIEF DESCRIPTION OF THE DRAWINGS

FIG. 1 is a block diagram of the detection flowchart of the present invention.

FIG. 2 is the expression levels of granulysin obtained by incubating lymphocytes from SCAR patients, tolerant controls or healthy donors with suspected drugs/metabolites for 1 to 2 weeks. Wherein, the expression of granulysin was determined by ELISA, and the fold change of granulysin was normalized by that of the solvent control.

FIG. 3 is the expression level of IFN-γ obtained by incubating lymphocytes from SCAR patients, tolerant controls or healthy donors with suspected drugs/metabolites for 1 to 2 weeks. Wherein, the expression of IFN-γ was determined by ELISA, and the fold change of IFN-γ was normalized by that of the solvent control.

FIG. 4 is the expression level of granulysin mRNA obtained by incubating lymphocytes from allopurinol-SCARs patients with drugs at concentrations of 1-fold (1×) or 10-fold (10×) the therapeutic levels for 1 week. Wherein, the expression of granulysin mRNA was determined by RT-PCR, and the fold change of granulysin was normalized by that of the solvent control.

### DETAILED DESCRIPTION OF THE PREFERRED EMBODIMENTS

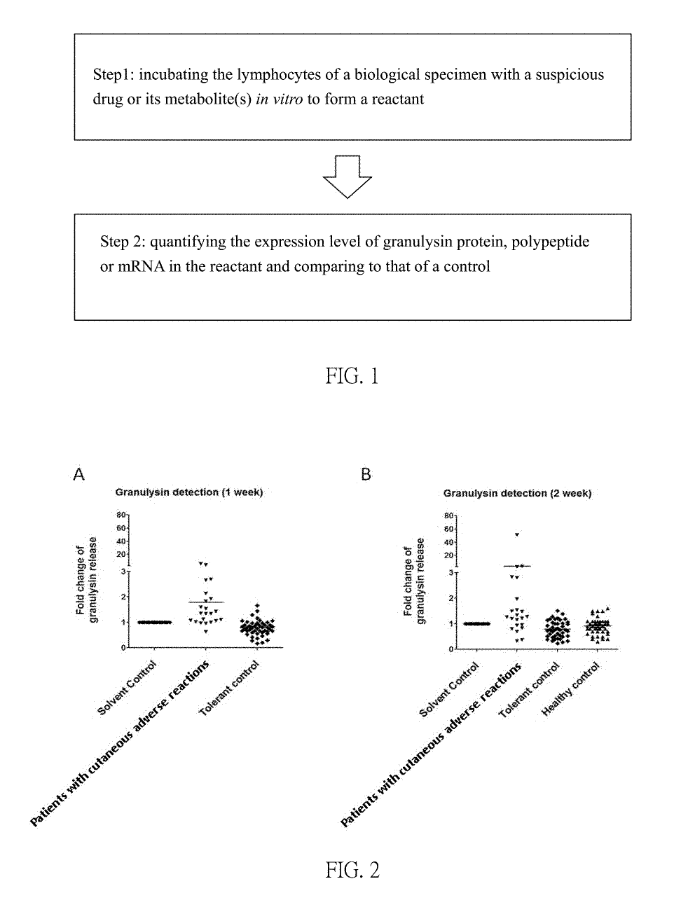

Referring to FIG. 1 through FIG. 4, the present invention relates to a method for identification of causative drugs of drug hypersensitivity reactions, comprising the steps of: step 1: incubating the lymphocytes from a biological specimen with the suspected drug or its metabolites in vitro to form a reactant; step 2: quantifying the expression level of granulysin protein, polypeptides or mRNA in the reactant and comparing it to that of the control. If the expression level of granulysin protein, polypeptides or mRNA is ≥1.2-fold higher compared to that of the control indicates the suspected drug or metabolites caused more lymphocyte activation and is the causative drug to probably induce drug hypersensitivity reaction.

The term “control” refers to the granulysin expression level of lymphocytes from biological specimens which are incubated in culture medium with or without the solvent for drugs.

Wherein, the expression level of granulysin in step 2 is quantified by capture antibodies and detection antibodies specific for the granulysin protein or the polypeptide in the reactant.

Wherein, the expression level of granulysin in step 2 is quantified by hybridizing specific oligonucleotide primers or probes to granulysin mRNA in the reactant.

Wherein, causative drug includes western drugs, Chinese medicine, vaccines or antigens which can induce T cell activation.

Wherein, the drug hypersensitivity reactions comprise Stevens-Johnson syndrome (SJS), toxic epidermal necrolysis (TEN), drug rash with eosinophilia and systemic symptoms (DRESS), maculopapular eruption (MPE), erythema multiforme majus (EMM), or fixed drug eruption (FDE).

The expression level of granulysin protein or nucleic acid in lymphocytes culture medium can be evaluated by obtaining the lymphocytes from a subject and culturing the lymphocytes with the suspected drug or its metabolites or the tolerant drug. The term “lymphocytes” includes cells from peripheral blood or biological fluids isolated from a subject, preferably from peripheral blood. Granulysin expression level can be measured in a number of ways, including, but not limited to: measuring the mRNA encoded by the granulysin gene; measuring the amount of protein encoded by the gene; or measuring the activity of the protein encoded by the gene.

The level of mRNA isolated from the cultured leukocytes in the reactant can be determined by hybridization or amplification assays that include, but are not limited to, Northern blot analyses, polymerase chain reaction analyses and probe arrays. One of the preferred diagnostic methods for the detection of the mRNA level involves using a nucleic acid molecule (probe) that can hybridize to the granulysin mRNA. The nucleic acid probe can be, for example, a full-length granulysin nucleic acid or a portion thereof, such as an oligonucleotide of at least 7, 15, 30, 50, 100, 250 or 250 nucleotides in length and sufficient to specifically hybridize under stringent conditions to granulysin mRNA or genomic DNA.

The granulysin mRNA level in the reactant also can be evaluated with nucleic acid amplification, e.g., by RT-PCR, ligase chain reaction, self-sustained sequence replication, transcriptional amplification system, Q-Beta Replicase, rolling circle replication, or any other nucleic acid amplification method, followed by the detection of the amplified primers using techniques known in the art. In the present invention, amplification primers are defined as being a pair of nucleic acid molecules that can anneal to 5′ or 3′ regions of a gene (plus and minus strands, respectively, or vice-versa). In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

In an embodiment of the present invention, the method comprises hybridization of a control sample with the amplification primers that detect granulysin mRNA or genomic DNA, and comparing the expression of granulysin mRNA or genomic DNA in the control sample and the test sample.

A variety of methods can be used to determine the level of granulysin protein in the reactant (the supernatant of the cultured lymphocytes). These methods comprise contacting an agent that selectively binds to the protein or its antigenic or immunogenic fragment, such as an antibody with a sample, and evaluating the level of protein in the sample. The techniques include enzyme linked immunosorbent assays (ELISA), enzyme linked immunospot (ELISPOT) assays, immunoprecipitations, immunofluorescence, enzyme immunoassay (EIA), radioimmunoassay (MA), and Western blot analysis.

In an embodiment of the present invention, the method further comprises the step of contacting the control sample with an antibody that detects granulysin to compare the expression of granulysin between the control sample and the test sample. This method further comprises a comparison of experimental values with reference values (e.g., incubation of the samples with the medium/solvent controls).

The present invention also comprises an identification kit for identification of the causative drug of drug hypersensitivity reactions, comprises the following reagents: a test kit to culture a reagent with lymphocytes from a biological specimen and with a suspected drug or its metabolite in vitro to form a reactant; a detection kit, to interact with the reactant in the test kit, and quantify the expression level of granulysin protein, polypeptides or mRNA in the reactant. If the expression level of granulysin protein, polypeptides or mRNA is ≥1.2-fold higher compared to that of the control, the suspected drug or its metabolite(s) could be determined as the causative drugs.

Wherein, the detection kit comprises oligonucleotide primers or probes which are specific for granulysin mRNA or genomic DNA to detect the mRNA expression level of granulysin in the reactant.

Wherein, the detection kit comprises a specific capture antibody and a detection antibody which are specific for granulysin to detect the expression level of granulysin in the reactant.

Wherein, the method for detecting the expression level of granulysin in the reactant by a capture antibody and a detection antibody is enzyme-linked immunosorbent assay (ELISA) or enzyme-linked immunospot (ELISPOT).

Wherein, the causative drug includes western drugs, Chinese medicine, vaccines or antigens which can induce T cell activation.

The methods described herein can identify the causative drug of a subject having, or at risk of developing the drug hypersensitivity reactions. As used herein, the term “drug hypersensitivity reactions” comprises maculopapular eruption (MPE), fixed drug eruption (FDE), erythema multiforme majus (EMM), severe cutaneous adverse reactions (SCAR), drug rash with eosinophilia and systemic symptoms (DRESS), Stevens-Johnson syndrome (SJS), or toxic epidermal necrolysis (TEN).

The method further comprises comparing the value of the test sample to the value of a control sample (e.g., incubation of the samples with the medium/solvent controls). The granulysin expression value of the samples can be obtained by any of the methods described herein, e.g., by providing a nucleic acid from the sample and contacting the nucleic acid to oligonucleotide primers or probes or contacting the supernatants or cells to the anti-granulysin antibodies.

The present invention comprises in vitro culture of lymphocytes with the suspected drugs and metabolites and detect the expression of granulysin protein by ELISA or ELISPOT, or the expression of granulysin mRNA by qualitative real-time PCR. The steps comprise isolate lymphocytes from the whole blood from a patient and culture the lymphocytes at 37° C. with 5% CO₂ in 96-well microplates in RPMI-1640 medium. Suspected drugs, drug metabolites or tolerant drugs to be co-cultured with the lymphocytes were diluted using the culture medium to obtain a concentration of physiologically therapeutic level (1-fold, 1×), 0.1-fold (0.1×), and 10-fold (10×), and co-culture for one to two weeks.

Incubation of lymphocytes with suspected drugs or metabolites: Peripheral blood mononuclear cells (PBMC) were isolated from the whole blood sample using Ficoll-Paque (Pharmacia Fine Chemicals, USA) density gradient centrifugation. PBMC (1.0×10⁶/well) were cultured in 96-well microplates in RPMI-1640 medium (GIBCO Invitrogen, Life Technologies, Carlsbad, Calif.) supplemented with 10% autologous serum and IL7 (1 ng/ml) at 37° C. in 5% CO₂ for one to two weeks. The suspected drugs, metabolites or tolerant drugs for examination were diluted in the medium to obtain a concentration reflecting the physiological therapeutic level (noted as 1-fold), 0.1-fold or 10-fold. For example, cells were incubated in the medium containing oxypurinol (10 μg/mL, 100 μg/mL) or tolerant drugs which had been used by the patients for more than 3 months without any adverse reactions. In addition, the solvent was added to the medium as the negative control, and phytohemagglutinin (PHA) of a concentration of 10 μg/mL was used as the positive control.

ELISA quantification of granulysin expression: Culture supernatants were collected on day 7 and day 14 for measuring the levels of granulysin using enzyme-linked immunosorbent assays (ELISA). Briefly, the plates (Nunc, Roskilde, Denmark) were coated with 50 μg/ml of anti-granulysin monoclonal antibody G011, and then blocked with 10% FBS in a washing buffer (PBS containing 0.1% Tween-20) and serially incubated reacted at room temperature with the blocking buffer for 2 hours; 1 μg/ml of biotinylated anti-granulysin monoclonal antibody G052, in the blocking buffer for 1 hour; and 2 μg/ml of horseradish peroxidase-conjugated streptavidin in the washing buffer. Between two incubations, the plates were washed using the washing buffer. Finally, the plates were incubated with the substrate solution containing H₂O₂ and tetramethylbenzinine with washing steps between each reaction. Samples were analyzed in triplicate. The assay sensitivity for granulysin was 1.56 ng/ml. In addition, the levels of IFN-γ in samples were measured using IFN-γ ELISA kits (Invitrogen, Carlsbad, Calif.) with a sensitivity of 1.56 pg/ml.

Quantitative Real-Time RT-PCR: Total RNA was isolated from the cultured leukocytes. The granulysin mRNA levels were quantified using a LightCycler (Roche molecular Biochemicals)-based, real-time, quantitative RT-PCR using the master SYBR Green 1 kit. The absolute copy number was determined using a method described previously. (Matsushita et al. Br J Haematol. 2001 March; 112(4):916-26). The following oligonucleotides were used:

granulysin:
5′-TCTCTCGTCTGAGCCC-3′
5′-GCAGCATTGGAAACACT-3′
β-actin:
5′-ACATCCGCAAAGACCT-3′
5′-AGGG TGTAACGCAACTA-3′

Statistical analysis: The fold-changes of granulysin in the culture supernatant of lymphocytes stimulated by the suspected drugs/metabolites or tolerant drugs were calculated by dividing the data by the solvent control. Significant difference between the groups was analyzed by one sample t test or student's t test. The sensitivity and specificity were calculated according to the standard definitions. All of the P values were two-tailed, and a P value of <0.05 was considered statistically significant. Statistical analyses were performed using the SPSS software Version 18.0 (SPSS Inc, Chicago, Ill.).

To validate if the granulysin could be useful for identification of causative drugs of drug hypersensitivity reactions, this example enrolled 22 patients with severe cutaneous adverse drug reactions (SCAR), including SJS, TEN, FDE, and EMM. PBMCs from these patients were isolated and cultured with the suspected drug or its metabolite in vitro for 1 to 2 weeks. The levels of granulysin and IFN-γ in the supernatants of cultures were measured by ELISA for comparing the sensitivity. The data shows that the sensitivity of granulysin is 77.3-81.8%. However, the sensitivity of IFN-γ is lower than 20% (Table 1). Therefore, the present experiment demonstrates that the detection of granulysin by ELISA in the granulysin-based drug lymphocyte stimulation test has a high sensitivity and can be used for identifying causative drugs of drug hypersensitivity reactions.

TABLE 1
Lymphocytes from twenty-two SCAR patients were stimulated
with the suspected drug for one to two weeks. The levels
of released granulysin and IFN-γ were measured
by ELISA to compare the sensitivity.

| | Sensitivity (%) | | |
|---|---|---|---|
| | Granulysin | IFN-γ | |
| 1 week | 77.3 | 18.2 | p = 2.03 × 10⁻⁴ |
| 2 week | 81.8 | 13.6 | p = 1.16 × 10⁻⁵ |

The present invention further examined the specificity of granulysin for identification of causative drugs of drug hypersensitivity reactions. To demonstrate that only the lymphocytes from drug hypersensitivity patients would induce granulysin expression when cultured with the causative drug/its metabolite, but not the lymphocytes from tolerant controls and healthy individuals, this example enrolled 11 tolerant controls and 10 healthy donors. Their PBMCs were isolated and cultured with causative drugs or metabolites in vitro for 1 to 2 weeks. The levels of granulysin and IFN-γ in the supernatants of cultures were measured by ELISA for comparing the specificity. The data shows that the specificity of granulysin and IFN-γ are 95.7% and 76.2% respectively after one week incubation in the tolerant controls. After two weeks incubation, the specificity of granulysin and IFN-γ are 92.9% and 77.8%, respectively in the tolerant controls (Table 2). In healthy donors, the specificity of granulysin and IFN-γ are 86.7% and 75.6%, respectively after two weeks incubation (Table 2). Therefore, this experiment demonstrates that the detection of granulysin by ELISA in granulysin-based drug lymphocyte stimulation test has a high specificity and can be used for identification causative drugs of drug hypersensitivity reactions.

TABLE 2
Lymphocytes from 11 tolerant controls and 10 healthy
donors were stimulated with drugs for one to two weeks.
The levels of released granulysin and IFN-γ were
measured by ELISA for comparing the specificity.

| Specificity (%) | |
|---|---|
| Tolerant control | Healthy control |

| | 1 week | 2 week | 1 week | 2 week |
|---|---|---|---|---|
| Granulysin | 95.7 | 92.9 | — | 86.7 |
| IFN-γ | 76.2 | 77.8 | — | 75.6 |

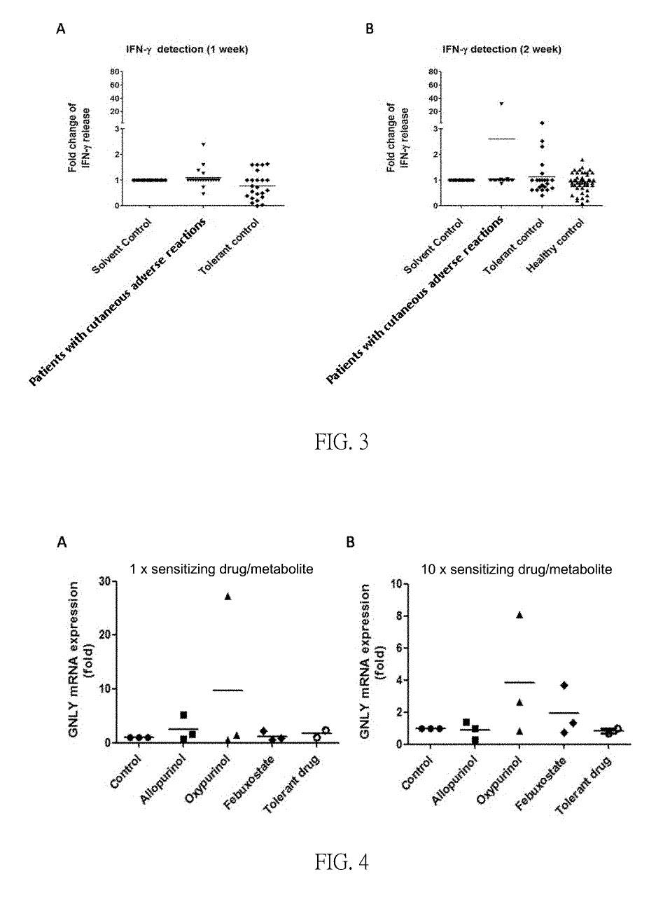

Lymphocytes from SCAR patients, tolerant controls and healthy donors were cultured with causative drugs/metabolites or tolerant drugs in vitro for one to two weeks, the expression of granulysin and IFN-γ measured by ELISA were shown in FIG. 2 and FIG. 3. The results show that robust expression of granulysin was induced in the cultures by incubating the causative drugs/metabolites with the lymphocytes from 22 SCAR patients. However, there was no statistical difference in tolerant controls and 10 healthy donors (FIG. 2 and FIG. 3). Although the expression of IFN-γ increased after lymphocyte stimulation in some SCAR patients (FIG. 3A and FIG. 3B), it was only limited to a few cases and was not sufficient as a specific biomarker. Therefore, the results suggest that the detection of granulysin by ELISA in granulysin-based drug lymphocyte stimulation test is the best method to identify causative drugs of drug hypersensitivity reactions.

FIG. 4 is directed to the granulysin mRNA levels. The present example enrolled three allopurinol-induced SCAR patients. Their lymphocytes were cultured with causative drugs/metabolites at concentrations of 1-fold (1×) or 10-fold (10×) or other tolerant drugs in vitro for one week. The expression of granulysin mRNA was measured by RT-PCR. The results also show that granulysin is more sensitive (66.7%) and more specific (100%), irrespective of whether it was stimulated by 1-fold (1×) or 10-fold (10×) causative drug/metabolite.

According to the above, the present invention proves that the granulysin-based T-cell activation assay could be a useful method for identifying causative drugs of drug hypersensitivity reactions with high specificity and sensitivity. The present invention provides methods for measuring granulysin mRNA and protein expression in an in vitro granulysin-based drug lymphocyte stimulation test. Also provided are kits for measuring granulysin mRNA and protein expression in an in vitro granulysin-based drug lymphocyte stimulation test, comprise the primers and probes to detect granulysin gene, and antibodies which can recognize granulysin protein.

While the means of preferred embodiments in present invention has been described herein, numerous modifications and variations could be made thereto by those skilled in the art without departing from the scope and spirit of the invention set forth in the claims. The modifications and variations should in a range limited by the specification of the present invention

1. A method for identification of a causative drug of a drug hypersensitivity reaction, comprising the steps of:
step 1: incubating the lymphocytes from a biological specimen with a suspected drug or its metabolite in vitro to form a reactant;
step 2: quantifying the expression level of a granulysin protein, polypeptides or mRNA in the reactant and comparing to that of a control,
wherein the expression level of the granulysin protein, polypeptides or mRNA is ≥1.2-fold higher compared to that of the control indicates the suspected drug or its metabolite is the causative drug.

2. The method of claim 1, wherein the expression level of granulysin level in step 2 is quantified by a specific capture antibody and a detection antibody to detect the granulysin protein or polypeptide in the reactant.

3. The method of claim 1, wherein the expression level of granulysin level in step 2 is quantified by hybridizing the granulysin mRNA in the reactant with specific oligonucleotide primer or probes.

4. The method of claim 1, wherein the lymphocytes are cells from peripheral blood or a biological fluid isolated from a subject, preferably from peripheral blood.

5. The method of claim 2, wherein the method of detection the expression level of granulysin in the reactant by a capture antibody and a detection antibody is enzyme-linked immunosorbent assay or enzyme-linked immunospot.

6. The method of claim 1, wherein the drug hypersensitivity reaction comprises Stevens-Johnson syndrome (SJS), toxic epidermal necrolysis (TEN), drug reaction with eosinophilia and systemic symptoms (DRESS), maculo-papular eruptions (MPE), erythema multiforme majus (EMM), and fixed drug eruption (FDE).

7. The method of claim 1, wherein the causative drug comprises western drugs, Chinese medicine, vaccines or antigens which induce T cell activation.

8. An identification kit for identification of a causative drug of a drug hypersensitivity reaction, comprising:
a test kit, to culture a reagent with lymphocytes from a biological specimen and with a suspected drug or its metabolite in vitro to form a reactant;
a detection kit to interact with the reactant in the test kit, and quantify the expression level of granulysin protein, polypeptides or mRNA in the reactant,
wherein the expression level of granulysin protein, polypeptides or mRNA is ≥1.2-fold higher than that of the control indicates the suspected drug or its metabolite is the causative drug.

9. The identification kit of claim 8, wherein the detection kit comprises a specific capture antibody and a detection antibody which are specific for granulysin to quantify the expression level of granulysin in the reactant.

10. The identification kit of claim 8, wherein the detection kit comprises oligonucleotide primers or probes which are specific for granulysin to detect the mRNA expression level of granulysin in the reactant.

11. The identification kit of claim 9, wherein the method for detecting the expression level of granulysin in the reactant by the specific capture antibody and the detection antibody is enzyme-linked immunosorbent assay or enzyme-linked immunospot.

12. The identification kit of claim 8, wherein the drug hypersensitivity reaction comprises Stevens-Johnson syndrome (SJS), toxic epidermal necrolysis (TEN), drug reaction with eosinophilia and systemic symptoms (DRESS), maculo-papular eruptions (MPE), erythema multiforme majus (EMM), and fixed drug eruption (FDE).

13. The identification kit of claim 8, wherein the causative drug comprises western drugs, Chinese medicine, vaccines and antigens which induce T cell activation.
